(19) United States
(12) Patent Application Publication
(10) Pub. No.: US 2018/0333205 A1
(43) Pub. Date: Nov. 22, 2018

(54) OPTICAL ENERGY DELIVERY AND SENSING APPARTUS

(71) Applicant: COOK MEDICAL TECHNOLOGIES LLC, BLOOMINGTON, IN (US)

(72) Inventor: Rune T. Paamand, Humlebaek (DK)

(21) Appl. No.: 15/983,170

(22) Filed: May 18, 2018

(30) Foreign Application Priority Data
May 18, 2017 (GB) 1707996.3

(51) Int. Cl. A61B 18/18; G02B 6/32; G02B 6/26; G01K 11/32

(52) U.S. Cl. CPC A61B 18/18; G02B 6/32; A61B 2018/00404; G01K 11/3206; G02B 6/262

(57) ABSTRACT

Optical energy delivery apparatus for ablation or embolization includes an optical fibre having a distal end which is provided a light directing element, such as a lens. The light directing element is configured to direct optical energy beyond the distal end of the optical fibre. The optical fibre includes at least one Bragg grating proximate the distal end for sensing a change in the optical fibre during its operation. The apparatus includes a control unit configured to drive an optical source and to obtain signals from a sensor unit. The controller may also drive the energy source at a sensing wavelength. The structure provides a single optical fibre supply optical energy and sense changes in optical fibre. The optical fibre may have a tapering diameter towards its distal tip for increased flexibility at its distal end.

### CROSS-REFERENCE TO RELATED APPLICATIONS

This application claims the benefit of priority under 35 U.S.C. § 119(a) to Great Britain Patent Application No. 1707996.3, filed May 18, 2017, which is incorporated by reference here in its entirety.

### TECHNICAL FIELD

The present invention relates to apparatus for delivering energy to a body part, and in the preferred embodiment to vascular optical energy delivery and sensing apparatus for carrying out blood embolization and/or vessel ablation.

### BACKGROUND ART

Conventional techniques for occluding a vessel include the use of implantable medical devices such as plugs and coils, particles such as GelFoam and PVA, or liquid embolic agents such as alcohol and Onyx. More recently, there has been research into heat induced embolization as an alternative to conventional embolization techniques. Heat induced embolization can avoid the problems of long term implantation of foreign objects in the body. However, electrosurgical devices suffer from disadvantages such as electrode charring, while small electrodes have limited power capabilities. Moreover, electrosurgical devices can suffer from control difficulties. For example, intrinsically, electrosurgical devices can only sense electrically derived characteristics such as impedance. As a result, it can be difficult to ensure that such devices reliably carry out the desired embolization or ablation, and also to do so without overheating the body part being treated or adjacent body parts. Overheating can lead to tissue or organ damage.

In order for an endoluminally deployable device to have wide applicability, it is important for it to have a small diameter and optimum flexibility, at least in a distal portion of the device. However, many electrosurgical devices have limited ability to be made with small diameters due to the multiplicity of components required in the devices.

Examples of known endoluminal ablation devices are described in U.S. Pat. No. 5,951,482, U.S. Pat. No. 9,149,333, US 2010/185,187, US 2013/090563, US 2014/288541 and WO 2015/074036.

### SUMMARY OF THE INVENTION

The present invention seeks to provide improved medical embolization and/or ablation and, particularly, improved optical energy delivery apparatus.

According to an aspect of the present invention, there is provided optical energy delivery apparatus for medical applications, including:

an optical fibre having a length, a proximal end and a distal end, the distal end including a light directing element configured to direct optical energy beyond the distal end of the optical fibre;

the proximal end of the optical fibre being configured to be coupled to a source of optical energy;

the optical fibre including at least one sensing lattice structure disposed therein at a position along the length thereof;

the optical fibre being a combined device for delivery of optical energy and for sensing changes in the optical fibre.

The structure provides a device able both to generate optical energy and to sense one or more parameters of the device, preferably in the course of its operation. Not only does this provide advantages in terms of sensing accuracy, but it also allows for a smaller diameter device able to navigate through and into smaller and more tortuous vessels.

Advantageously, the at least one lattice structure is an inscribed micro-lattice structure.

Preferably, the at least one lattice structure is a Bragg grating.

In the preferred embodiment, the apparatus includes a plurality of lattice structures disposed at spaced intervals along the length of the optical fibre.

The light directing element at the distal end of the optical fibre may be or may include a lens. A lens is able to focus light energy to intensify the energy at a chosen focal point, or to disperse energy, for instance to reduce heating power and/or to treat a wider area.

The lens may be a concave lens. In other embodiments, the lens may be a concave axicon lens. A practical embodiment has a lens which is a combination lens in the form of one of: a double convex axicon lens and a double concave axicon lens.

The light directing element at the distal end of the optical fibre may be or may include at least one angled surface configured to direct light at an angle out of the distal end of the optical fibre. In some embodiments, the light directing element includes at least two angled surfaces, configured to direct light in at least two directions out of the distal end of the optical fibre. Advantageously, the or each angled surface directs light forwardly and radially out of the distal end of the optical fibre.

In some embodiments, the optical fibre has a varying diameter along its length. Similarly, in some embodiments the optical fibre has a varying stiffness along its length. For this purpose, the optical fibre may have a reducing diameter in the direction of its distal end. In an example, this may be a tapering diameter along it's the whole or a part of its length in the direction of its distal end. In other embodiments, the optical fibre has a distal end portion with a smaller diameter relative to the diameter of a proximal portion of the optical fibre.

It is also envisaged that the optical fibre may include a stiffening structure in a proximal portion thereof. A stiffening structure of such a nature can provide a change in stiffness of the optical fibre over its length.

Typically, the optical fibre is preferably more flexible is a distal section relative to a proximal section thereof.

The apparatus advantageously includes a source of optical light. In other embodiments, the apparatus may be connectable to a separate light source via a suitable coupling, of which many types are known in the art.

In the preferred embodiment, the apparatus includes a sensing unit operable to detect changes in light reflected from the at least one lattice structure and to determine therefrom changes in at least one of: temperature, stress and strain in the optical fibre. These parameters can be used to obtain an indication of embolization and/or ablation progress.

In an embodiment, the proximal end includes a hub for connection to the source of optical energy.

Advantageously, the apparatus includes a control unit coupled to the source of optical energy and operable to drive the source so as to generate light at a plurality of wavelengths. In preferred embodiments, the control unit is operable to utilize different wavelengths for different functions. For example, it may be configured to use a first generated wavelength for power transfer. A suitable first wavelength may be between 600 nm and 1400 nm; advantageously around 970 nm. The control unit is preferably also configured to use a second generated wavelength for sensing. The second generated wavelength is advantageously different from the first generated wavelength and is advantageously in the region of 1550 nm, although it could also be or additionally be around 800 nm. Use of different wavelengths provides a robust system for delivery of optical energy and for sensing changes within the same optical fibre.

According to another aspect of the present invention, there is provided a method of delivering optical energy to an organ of a patient by means of apparatus including an optical fibre having a length, a proximal end and a distal end, the distal end including a light directing element configured to direct optical energy beyond the distal end of the optical fibre; the optical fibre including at least one sensing lattice structure disposed therein at a position along the length thereof and being a combined device for delivery of optical energy and sensing changes in the optical fibre; the method including the steps of: generating and passing optical energy in the fibre at a first wavelength, the first wavelength being a heating wavelength for said organ; generating and passing optical energy in the fibre at a second wavelength, said second wavelength being a reflection wavelength of the lattice structure; and determining from the reflection wavelength an indication of temperature of the optical fibre.

Preferably, the step of determining an indication of temperature of the optical fibre determines an amplitude of the reflection wavelength. The step of determining may be operable to determine changes in at least one of: temperature, stress and strain in the optical fibre.

Advantageously, the optical fibre includes a plurality of lattice structures disposed at spaced intervals along the length of the optical fibre, the method including the step of determining an indication of temperature at each of the lattice structures.

In one embodiment, optical energy is generated simultaneously at the first and second wavelengths. In another embodiment, optical energy is generated in multiplexed form at the first and second wavelengths.

Other aspects and advantages of the teachings herein are described below in connection with the preferred embodiments disclosed herein.

### BRIEF DESCRIPTION OF THE DRAWINGS

Embodiments of the present invention are described below, by way of example only, with reference to the accompanying drawings, in which:

FIG. 1 is a schematic diagram of the preferred apparatus in situ in a vessel;

FIG. 2 is a schematic representation of a concave lens for diverging light towards the vessel wall;

FIG. 3 is a schematic representation of a concave axicon lens for focusing light at the vessel wall at a distance;

FIG. 4 is a schematic representation of a double concave axicon lens for focusing diverging light in an annular ring;

FIG. 4A is an enlarged view of the double concave axicon lens of the embodiment of FIG. 4;

FIG. 5 shows the principles of a fibre Bragg grating;

FIG. 6 is a schematic representation showing the characteristic wavelength of the change in period caused by changes in physical properties of the fibre;

FIG. 7 is an absorption spectrogram of blood and tissue at various wavelengths;

FIG. 8 is a graph reflecting light reflection by a Bragg grating when subjected to a physical change such as strain and/or a temperature change;

FIG. 9 is a schematic representation of a fibre guide wire with a convex axicon lens and fibre Bragg grating zones along the length of the fibre;

FIG. 10 is a schematic representation of a fibre guide wire with a double concave axicon lens and fibre Bragg grating zones along the length of the fibre; and

FIGS. 11 and 12 show different examples as to how to vary the flexibility of a fibre according to the teachings herein along its length.

### DESCRIPTION OF THE PREFERRED EMBODIMENTS

It is to be understood that the drawings are schematic only and not to scale. Often, only the principal components of the apparatus relevant for an understanding of the teachings herein are shown in the drawings and for the sake of clarity inessential elements commonplace in the art are not shown.

The embodiments described below relate to apparatus which can be passed from a remote percutaneous entry point endoluminally through the vasculature of a patient up to the point of treatment. The apparatus preferably has a small diameter at least at its distal end so as to be able to pass through and into small diameter vessels.

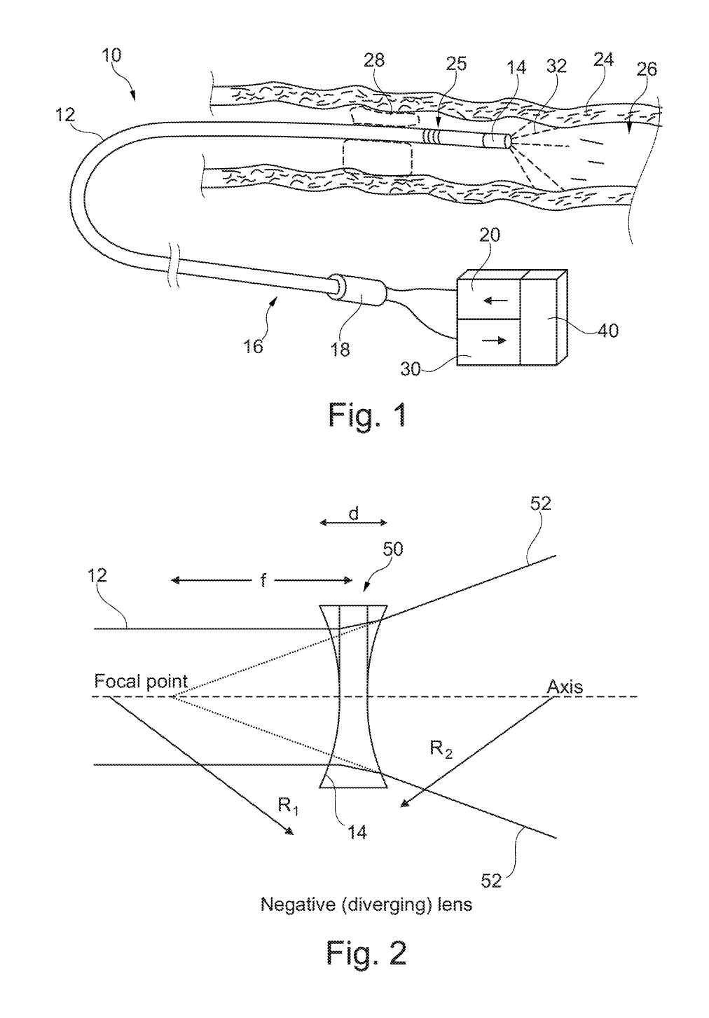

The apparatus may be used for embolization of blood, that is to create a blood clot which will then occlude a vessel. In other embodiments, the apparatus is configured to ablate a vessel, typically by heating the vessel wall at a plurality of discrete points around a circumference of the vessel or entirely around the vessel's circumference. Ablation causes contraction of the vessel and fusing of the vessel wall in a closed state, thereby to block the vessel. The apparatus can also be configured, by provision of suitable light directing elements at the distal end of the optical fibre, so as to carry out both ablation and embolization. Vessel ablation is generally considered better than blood embolization, for the reason that in the course of events the flow of blood will act to draw away heat generated by the device and therefore to temper the procedure. Where it is desired to use the apparatus to cause blood embolization, there can be provided a temporary vascular plug, such as the balloon shown in dotted outline in FIG. 1.

In the case of vessel ablation, the system is preferably set up to heat and destroy the inner lining of the vessel wall, that is the endothelium of the tunica intima. For this purpose, a relatively low power is required, typically of less than 5 Watts.

Referring first to FIG. 1, this shows in schematic form the principal components of an embodiment of apparatus 10 according to the teachings herein. The apparatus 10 includes an optical fibre 12 having a distal end 14 terminated with a light directing element configured to direct optical energy beyond the distal end 14, and of which various embodiments are described below. At the proximal end 16 of the optical fibre 12 there is provided a coupling hub 18 which is configured to be coupled to a source 20 of optical energy, in the preferred embodiment a light source of wide-band wavelength such as a super luminescence diode. The hub 18 is also coupled to a sensing unit 30 for purposes which are described in further detail below. Suitable sensing units include spectrum analysers (polychromators) or other photosensitive devices such as photodiodes or photoresistors with suitable wavelength sensitivity (as determined by the FBG wavelength). A controller 40, which is typically a microprocessor based controller, is coupled to the source 20 of optical energy and to the sensing unit 30. The controller 40 is configured to control the operation of the apparatus 10 in the manners described below.

The optical fibre 12 typically includes a core of glass or silica surrounded by a protective sheath. For the purposes of ablation and/or embolization the optical fibre preferably has a diameter of between 0.25 mm to 1.0 mm. The diameter of the fibre is related more to the diameter of the vessel to be treated.

FIG. 1 and the other embodiments depicted in the drawings show a fibre which has a generally straight distal end. In some embodiments, however, the fibre distal end may be curved, for example into a J-shape. This can assist in guiding the fibre through tortuous vasculature and into branch vessels, as may also assist in orienting the fibre in cases orientation is desired.

In FIG. 1, the apparatus 10 is shown schematically in an in-use configuration, in which the proximal end 14 of the optical fibre 12 is positioned within a blood vessel 24 of a patient. For this purpose, the optical fibre 12 will typically be fed through a catheter of an introducer assembly, the latter optimally being deployed via the Seldinger or a similar technique. The catheter and other components of the introducer assembly which will typically be used are not depicted in FIG. 1 for the sake of clarity.

The distal end 14 of the optical fibre 12 is shown positioned generally in the centre of the lumen 26 of the vessel 24, and this may be achieved in any conventional or suitable manner, for instance by means of a supporting catheter, a supporting balloon disposed around the catheter, and so on. The dotted outline 28 shown in FIG. 1 depicts a positioning balloon, which in practice would be attached to a delivery catheter within which the optical fibre 12 is located. A suitable balloon could be doughnut shaped or have the conventional elongate cylindrical form and is inflatable and deflatable via a suitable supply lumen in the catheter. In other embodiments, a balloon of this nature could be fitted to an outer sheath forming part of the optical fibre 12. The skilled person will appreciate that there are other methods of locating the fibre 12 in the centre of a vessel, including for instance support legs and so on. It should be understood, though, that it is not necessary to centre the fibre distal end 14 in the vessel as it will function effectively even when close to or abutting one side of the vessel wall. In such a circumstance, the vessel wall part close to the fibre tip will be heated more by the optical energy than the more remote parts of the vessel wall, which can may result in that part of the vessel wall being affected first, leading to differential swelling or radial contraction of the vessel, which will push the fibre closer to the centre point and to the other wall sections. This is not considered to be a disadvantage in may applications. It has been found that it may typically take around 15 seconds for a part of a vessel to react to such heat energy and contract.

FIG. 1 also shows, by way of the dotted lines 32 the directions of optical energy emanating from the light directing element of the optical fibre 12. The lines 32 depict a variety of possible options. For example, the light directing element could include a convex lens intended to focus light energy at a focal point beyond the extremity of the distal end 14, into the middle of the vessel 24, in order to heat and as a result cause blood embolization. In other embodiments, the light may be directed forwards and outwardly of the distal end 14 by the light directing element, typically in the form of a cone of light, to heat the vessel wall 24 for the purposes of ablation. Examples of suitable light directing elements for this purpose are given below.

Referring now to FIGS. 2 to 4, these show different embodiments of light directing element formed at or attached to the tip 14 of the optical fibre 12. Considering first FIG. 2, this shows in schematic form a concave lens 50, which would in practice be disposed at the extremity of a convex curved tip 14 of the optical fibre 12. A concave lens of this nature would cause light from the optical fibre 12 to diverge radially outwardly and forwardly, in a conical envelope, as depicted by the diverging lines 52 in FIG. 2, at an angle dependent upon the radii of curvature r1 and r2 of the two sides of the lens 50. The skilled person will appreciate that the ranges of curvature of the lens 50 can be varied in order to vary the angle of spread of the light beam and as a result the position of ablation/embolization relative to the tip 14 of the optical fibre 12. A wider angle dispersion cone produced by a smaller radius of curvature of the lens 50 will result in a shorter distance of travel of the light through the blood plasma and this will impinge over a shorter length of vessel wall, thereby applying greater energy to the vessel wall, whereas a shallower angle dispersion cone produced by a greater radius of curvature of the lens will result in the light travelling a greater distance through the blood plasma and in the beam heating a greater length of vessel wall. The energy applied per unit area of vessel wall will therefore be less. In practice, the optimum radius of curvature of the lens 50 will be determined by the vessel type, typically size and wall structure, and the nature of the blood flow in the vessel, which will affect heating efficiency. These are parameters which can readily be derived by routine testing and apply to all the embodiments disclosed herein.

The embodiment of FIG. 2 shows a lens 50 which is concave at both of its surfaces. In other embodiments, the lens could have a flat rear surface, to abut a flat fibre tip 14, and a concave front surface.

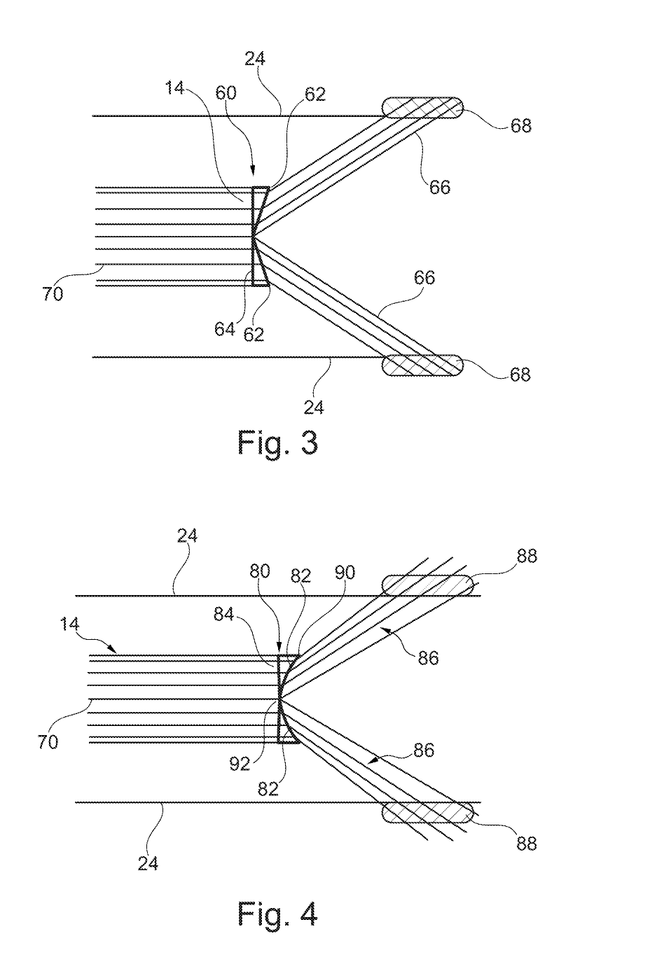

Referring now to FIG. 3, this shows another embodiment in which the light directing element is a concave axicon lens 60 disposed at the extremity of the tip 14 of the optical fibre. The axicon lens 60 has a surface 62 of concave conical form, that is a rotationally symmetric concave prismatic surface, which causes light 70 from within the optical fibre 12 to spread forwardly and radially outwardly from the surface 62, into a cone of parallel beams 66, as depicted in FIG. 3. The conical diverging beam 66 impinge upon the vessel wall 24 within a zone 68, which in practice will be the circumferential zone of the vessel wall. As the beam 66 of the diverging light cone does not spread, that is the beams in the radial direction are parallel, consistent with the straight radial surface of the lens 60, the length of the zone 68 will be directly proportional to the radius of the surface 62.

As the skilled person will appreciate, the angle of the conical surface 62 relative to the flat fibre tip 14, and equally to the back surface 64 of the lens 60, can be varied in order to vary the angle of divergence of the beam 66. This affects, as a result, the point of impingement of the beam 66 onto a vessel wall 24 relative to the distal end of the fibre assembly, and also the length (in the longitudinal direction of the vessel 24) of the zones 68 which are subjected to the optical energy.

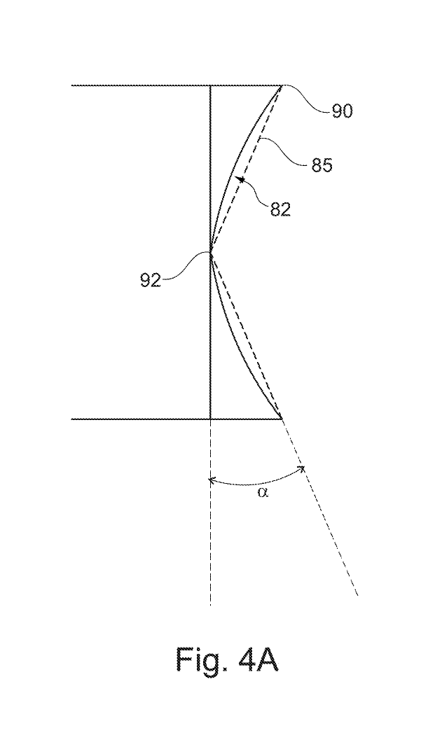

Referring now to FIG. 4, this shows another embodiment, in which the light directing element is a double concave axicon lens 80 having a planar rear surface 84 and a double concave axicon surface 82 at its front. The lens 80 is coupled, as with previous embodiments, to the front, flat, surface of the optical fibre tip 14. The double concave axicon surface 82 disperses light conically from the optical fibre 12 into a ring-shaped light beam 86, similar to the embodiments of FIGS. 2 and 3. However, in this instance each beam portion 86 has a diverging radial spread, as shown in FIG. 4, such that the zone 88 of the vessel wall is longer than the zone 68 produced by the axicon lens 60 of the embodiment of FIG. 3. This is achieved by the overall angle α of the surface 82 (see FIG. 4A) and by the curving convex shape of each side of the surface 82 relative to a straight line 85 (again as shown in FIG. 4A. This shape is useful in spreading the optical energy along the greater length of the vessel wall 24 and also in reducing the intensity of the optical energy reaching the vessel wall.

The optical fibre 12 has incorporated within its structure a sensing device, in this case a Bragg grating. In practice, this may be an inscribed micro-lattice structure formed into the surface of the fibre 12 in known manner. In one example this may be by UV light inscription into a UV sensitive fibre core (for instance made of silica) by laser light or through a mask, so as to form a series of regular circumferential rings spaced by a given spacing (desired reflection wavelength) relative to one another. It is to be understood, though, that the Bragg grating may be formed in different manners, one example being by way of a sandwich structure of different materials at the intended location of the or each grating.

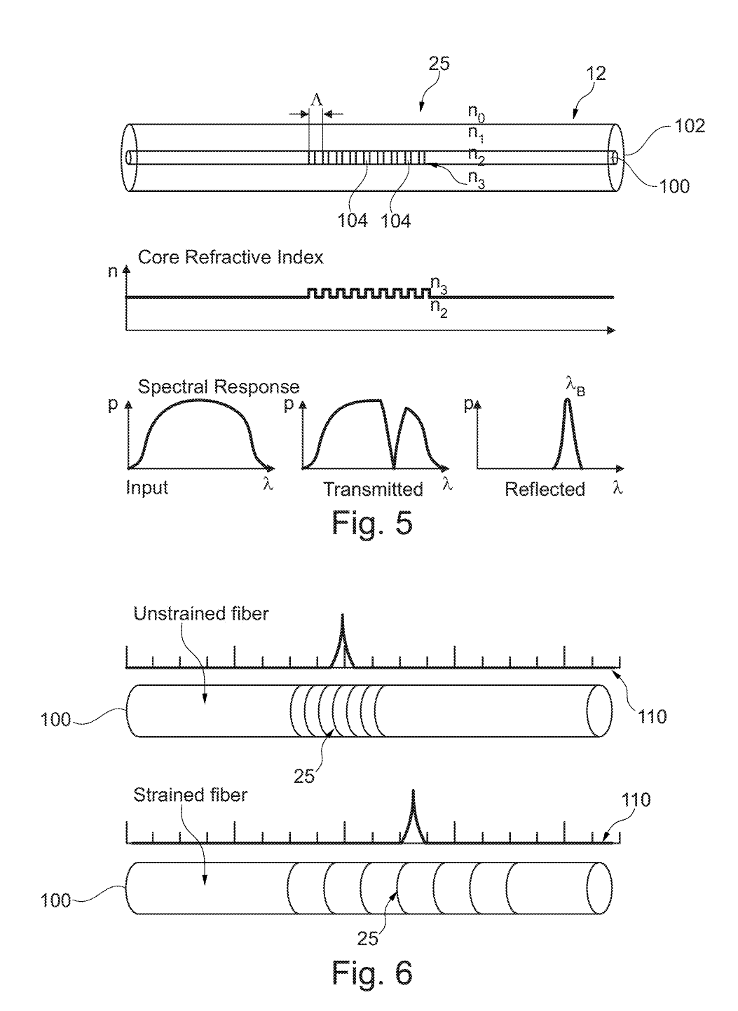

With reference to FIG. 5, the optical fibre 12 is shown with the sheath or cladding 102 enveloping the fibre core 100, as explained above. The Bragg grating 25 is formed within the fibre core 100 and comprises a plurality of spaced grating rings 104 at a given spacing equivalent to the wavelength of the selected reflection frequency of the grating, which provides a periodic variation in the refractive index of the fibre to act as a wavelength specific mirror. With reference also to FIG. 6, this shows the effect on the Bragg grating 25 caused by a physical change in the fibre core 100, in this case an elongation of the fibre core, for instance by pulling, or by a change in temperature or pressure, which may equally result in elongation of the fibre core 100. The skilled person will appreciate that the difference between the unstrained fibre and the strained fibre shown in the two sketches of FIG. 6 is exaggerated, for the purpose of clarity. The change in spacing between the Bragg gratings when the fibre is elongated will cause a shift in reflected wavelength which will provide a direct indication of the change in the associated characteristic of the fibre. Once the optical fibre 12 has been positioned within a vessel 24 of a patient, a measure of the reflected wavelength from the Bragg grating 25 can be obtained and this can be measured at intervals, for instance during the delivery of optical energy through the fibre 12, which will be indicative of a change of temperature in the fibre 12. That change in temperature will be caused by heating of the surrounding tissue and/or blood and can provide a reliable indication of temperature change within the organ being treated. A correlation between temperature and change in reflected wavelength form the Bragg grating 25 can be determined empirically by a suitable calibration routine and stored in a memory of the processor 40, for instance as a formula or as a look-up table.

More specifically, a change in temperature of the fibre 12 will be caused by heating of the surrounding tissue and/or blood and this can as a result provide a reliable indication of temperature change within the organ being treated. A correlation between temperature and change in reflected wavelength from the Bragg grating 25 is given in equation 1 below. It can be taken that there will be no noticeable change in strain during the change in temperature, as the fibre 12 would not normally be moved during the ablation procedure or during each stage of the ablation procedure if carried out in multiple stages. In light of this, the equation linearly relates the measured change in wavelength to the temperature given the known constant coefficients, and the temperature can be determined by a suitable calibration routine with coefficients stored in a memory of the processor 40, for instance as a formula or as a look-up table.

$$\frac{Δλ}{{λ}_{0}} = ( 1 - {p}_{e} ) \cdot ɛ + ( {α}_{Λ} + {α}_{n} ) \cdot Δ T ; \qquad ( eq . 1 )$$

$$\frac{Δλ}{{λ}_{0}}$$

Relative wavelength shift
Pₑ: Strain-optic coefficient
ε: Strain
α_{Λ}: Thermal expansion coefficient
αₙ: Thermo-optic coefficient

In cases where is can be expected that strain will not be not constant during the ablation procedure, that is during the period of change in temperature, two or more fibre Bragg gratings may be used, physically positioned so that they either experience same change in temperature with varying strain or same strain with varying change in temperature. The temperature can then be derived by solving the linear system of two equations with two variables.

Referring to FIG. 8, this shows the effect on reflected light caused by changes in the spacing between the Bragg rings 25 when the fibre is subjected to a physical change such as strain and/or temperature change. If graph A is taken to be representative of the normal reflection curve over frequency for a grating, graph B can be deemed representative of a change in the spacing of the Bragg rings. In this example, the curve B is down in frequency, as would occur when the fibre is stretched by strain or heating, for example. The frequency shift can be measured in one of two ways. First, this can be by identifying the change in intensity of the reflected light at a given frequency, depicted by 11 and 12 in FIG. 8. Another method is by looking for the frequency at which there is maximum intensity, for instance F1 and F2. Of course, intensity at a frequency may change as a result of either elongation of contraction. However, since the fibre is only ever likely to be stretched or heated, it is not necessary for the system to be able to identify in which way the nominal frequency of reflection has shifted. It is only the amount of shift which is relevant, as this will in use be indicative of the amount of heating of the fibre 12.

It will be appreciated that different sets of Bragg gratings 25 can be configured to reflect different, non-overlapping frequencies so as to produce a plurality of such intensity curves.

As a result, the sensing unit 30 and processor 20 may be configured to measure intensity or maximum frequency for each set of Bragg gratings, as described above in connection with FIG. 8.

In practice, once the fibre 12 has been positioned in a patient at the vessel location to be treated, the fibre is unlikely to incur any change further change in its state of strain. As a result, once positioned, the processor 40 can obtain a reference measurement from the or each Bragg grating 25 prior to initiation of the ablation or embolization process, that is prior to the generation of heat energy within the vessel. Heating will then be determined from a change in wavelength of the reflected light form the Bragg grating or gratings 25 relative to the reference measurement.

It is envisaged that in some embodiments there could be provided at least one second fibre (not shown in the drawings), also provided with one or more Bragg gratings, optimally adjacent the Bragg grating or gratings of the first fibre 12. This at least one second optical fibre can be used to obtain a second indication of strain for use in calibrating the measurements in light of overall strain. In practice, the second fibre can be arranged to incur the opposite strain to the first fibre 12, such as a compressive strain when the first fibre 12 incurs a stretching strain or vice versa, and thus provide a balancing reference measurement. This can be particularly useful for assemblies which have a curved, such as a J-shaped, distal end when unbiased.

In a practical embodiment, there may be provided a plurality of sets of Bragg gratings 25 at the distal end of the fibre 12 spaced from one another by, for example, by around 1 mm to around 5 mm. A plurality of sets of Bragg gratings can be used to measure different temperatures but also, or in the alternative, to measure the progress of the procedure for occluding the vessel. For instance, while the vessel is open and blood can flow therethrough, the blood will have a cooling effect such that it could be expected that there will be a temperature difference between different Bragg gratings. On the other hand, once the vessel has closed, there is no further blood flow to cool the fibre and the temperature measurement at the grating or gratings further down the fibre, in the proximal direction, will sense a rise in temperature which will be indicative of the successful occlusion of the vessel and therefore the end of the procedure. Thus, a plurality of sets of Bragg gratings 25 can provide a measure of operating characteristics along the length of the fibre 12. In some embodiments, though not necessary, there may be provided one or more Bragg gratings more proximally along the fibre relative to the zone at which ablation is effected, so as to obtain a measure of body temperature for use as a reference value. It is also not excluded that a proximal set of Bragg gratings may be provided to obtain a measure of ambient temperature.

It is to be appreciated that the sets of Bragg gratings spaced along the length of the fibre, for example each of the sets, can include groups of two or more lattice structures physically positioned so that they either experience same change in temperature with varying strain or same strain with varying change in temperature as discussed above, for example so that the temperatures at each of the lattice structures can be derived by solving the linear system of two equations with two variables. For example, each of the sets can include groups of two or more lattice structures physically positioned so that they experience same change in temperature with varying strain.

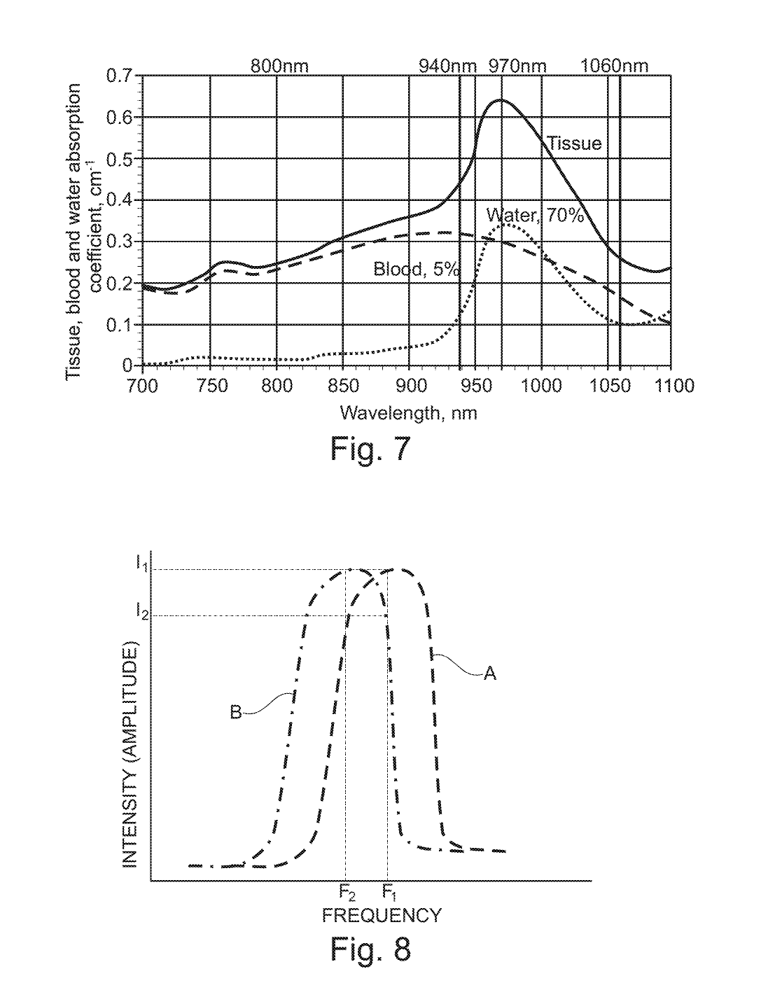

The apparatus 10, and in particular the control unit 40, may utilise several different wavelengths for different functions. For example, one wavelength may be selected for power transfer (for instance for embolization or ablation), which may be selected based on the absorption coefficient of blood and/or tissue. This has the advantage of targeting the delivery of power to the material intended to be ablated or caused to embolize, while minimising power delivery to other organs. With reference to FIG. 7, this shows an absorption spectrogram for blood, water and tissue. It is preferred that the apparatus 10 utilises a wavelength between 600 nanometres and 1400 nanometres, although for ablation of vessel tissue it is preferred that this wavelength be in the region of 970 nanometres. For embolization of blood, this wavelength will be optimally in the region of 940 nanometres, but typically within a range of around 800-1000 nanometres. Where it is desired to heat a plurality of different materials, for example tissue and blood, the control unit 40 may operate to generate a plurality of different heating wavelengths by suitable control of the optical energy source 20.

In a practical example, the control unit 40 is configured to generate in the region of 5 Watts or less of energy over a time period of less than 1 minute and preferably of between 5 to 30 seconds. Typically, though, the amount of energy to be generated and the length of time over which this should be supplied is dependent on the nature of the vessel and the of blood flow through the vessel. A vessel with thicker vessel walls will need greater energy to be ablated, compared to a smaller vessel or one with more delicate vessel walls. Similarly, a vessel experiencing higher blood flow will require the provision of more optical energy compared to a vessel experiencing lower blood flow. These parameters can be determined by the skilled person empirically by routine development processes.

The control unit 40 is also configured to operate the device at other wavelengths, or a spectrum of wavelengths, for use by the sensor unit 30 to detect the operating state of the optical fibre 12. These wavelengths will typically fall outside the range of wavelengths which would be absorbed by tissue or blood. For this purpose, in the preferred embodiments, the Bragg grating or gratings 25 have reflective wavelengths beyond the sensitive wavelengths of the vessel wall or blood and in the preferred embodiment around 1550 nm. Where a plurality of sets of Brag gratings 25 is provided, these may be spaced from one another in terms of wavelength by around 10 nm or more and in some cases up to around 25 nm. The wavelength spacing allows the sensor unit 30 to determine the status of each Bragg grating without interference, and preferably also without aliasing artefacts, from the other Bragg gratings in the fibre 12 and also allows for variation on the reflected wavelengths caused by strain or temperature changes in the fibre at the location of each Bragg grating 25.

In other embodiments, one or more Bragg gratings 25 may be formed to reflect light of around 800 nm wavelength.

The wavelengths used for sensing the state of the fibre 12 may be emitted at low power levels and the reflection pattern used to detect physical changes at the fibre Bragg grating zones 25. In one embodiment, two or a few specific wavelengths may be used instead of a spectrum, and the relative reflected intensities of these wavelengths used to detect physical changes at the zones of each Bragg grating 25

The power of light generated by the light source 20 for use in sensing is preferably in the region of 10 mW or less.

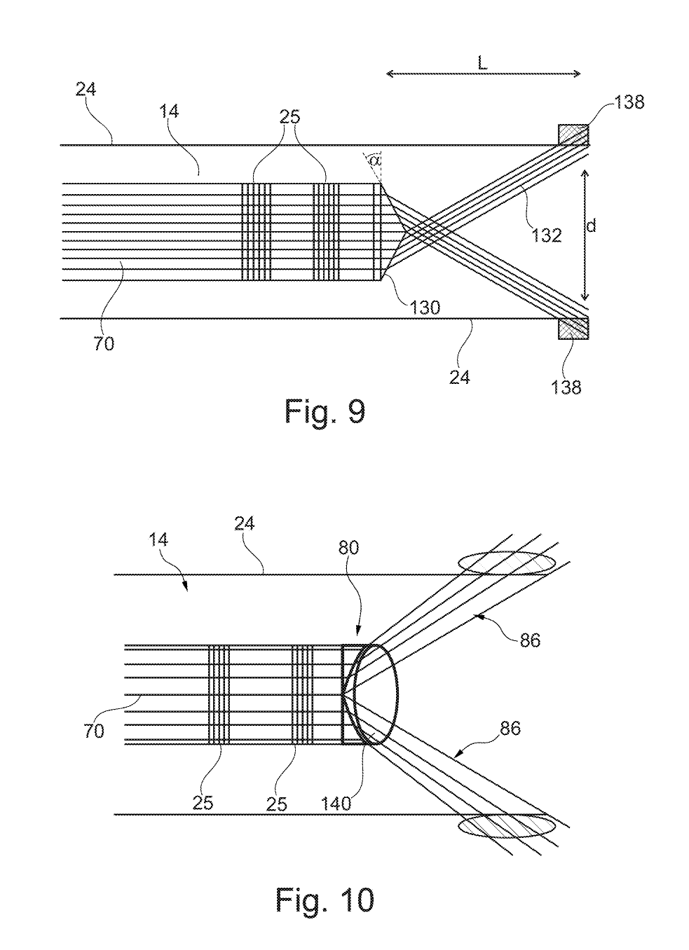

With reference now to FIG. 9, this shows the distal end 14 of a preferred embodiment of optical fibre 12 assembly, which includes a convex axicon lens 130 disposed at the fibre tip 14 and a plurality of Bragg grating zones 25 spaced from one another and close to the extremity of the distal fibre end 14. The lens 130 causes dispersion of the light 70 into a conical light beam 132, which impinges onto the vessel wall 24 tissue in an annular heating zone 138. The skilled person will appreciate that in place of a convex axicon lens there could be provided a concave axicon lens or other lens, for instance of any of the types depicted in FIGS. 2 to 4.

The Bragg gratings 25 may be configured to reflect the same wavelength or different wavelengths. These different wavelengths can be used for measuring changes during different modes of operation, for instance the state of the fibre 12 during different heating modes, such as heating tissue, heating blood, heating a combination of the two, and so on.

FIG. 10 shows another embodiment similar to that of FIG. 9 but in which in place of the convex axicon lens 130 there is provided a double concave axicon lens 80, as in the embodiment shown in FIGS. 4 and 4A. In addition, in this embodiment there is provided an atraumatic tip 140 in the form of a volume of silica or similar, that is a soft translucent or transparent material with an index of reflection preferably very close to or identical to blood, which avoids having to leave exposed the sharp edge of the concave lens. In all other respects, the embodiment of FIG. 10 has the same features as that of FIG. 9. It will be appreciated that a concave axicon lens will not produce an energy focal point inside the vessel as can occur with a convex axicon lens.

As will be particularly apparent from FIGS. 9 and 10, the structure provides a single fibre which can be used both for delivery of optical energy and for sensing change within the fibre 12 caused by changes in the surrounding material at the fibre tip 14. The structure can therefore be made with a very small diameter and as a result able to be passed through and into small diameter vessels. The structure also reduces the relative stiffness of the apparatus, particularly in comparison to prior art structures. Furthermore, the provision of a Bragg grating 25 in the same optical fibre 12 as that used for energy delivery provides a direct indication of changes in state of that optical fibre. The provision of a plurality of Bragg gratings can be used for sensing temperature and/or pressure, which can be inferred by the control unit 40 by measuring the shift in reflected wavelength. As explained above, wavelengths may be selected on the basis of the absorption co-efficient of the material sought to be heated and to minimise power delivered to other materials or organs. For instance, a wavelength may be chosen to heat tissue and minimise, to the extent possible, heating of blood, and vice versa.

The control unit 40 may be configured to deliver optical energy and to sense simultaneously, making use of different optical frequencies for the purpose, but can equally be configured to operate in a multiplexed form, that is to cycle between heating and sensing. In practice, there may be provided a plurality of sensor modules or inputs, each tuned to a particular Bragg grating, in its expected range of operation. A plurality of sensor modules allow for continuous operation. However, a single sensing module 30 with a single input may be provided working in a multiplexed manner. The inventor has discovered that the apparatus can be operated at a multiplexing time frame which is much shorter that the time taken for any changes to be experienced in the surrounding blood, so any interruption in energy delivery is not expected to make any practical difference to the ablation process.

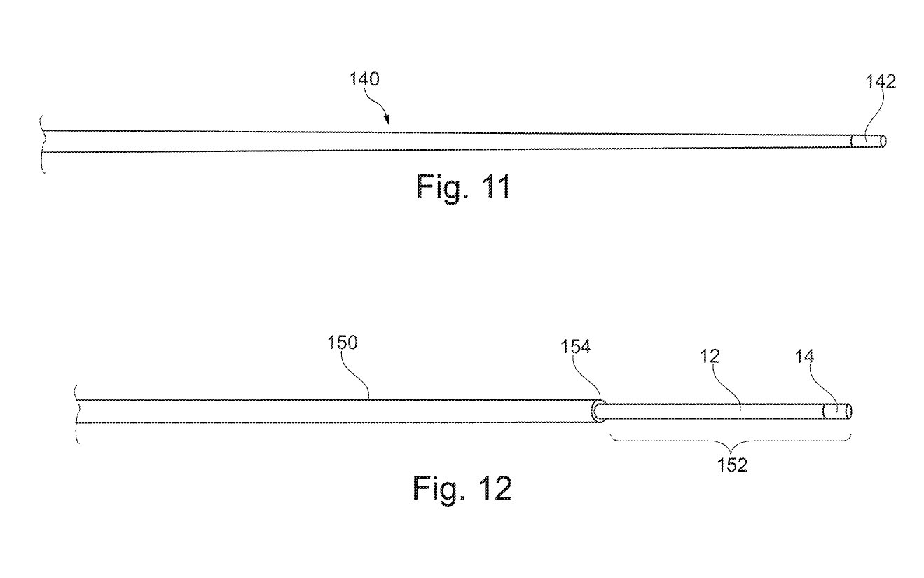

With reference now to FIG. 11, this shows another embodiment of optical fibre 140 which tapers along its length, so as to alter the rigidity (durometer) of the fibre, specifically towards the distal end 142 of the fibre 140, typically to ensure that the fibre becomes more flexible towards its distal end 142. In practice, the taper may be provided in the sheath or cladding of the fibre rather than the inner core. Having a more flexible distal portion assists in feeding the optical fibre 120 through delicate and tortuous vessels.

The fibre 140 may taper throughout its length or may taper for only a portion of its length, in which case it would have a proximal portion of substantially uniform diameter and then a distal portion of reducing diameter towards the distal tip 142. It is preferred that the taper is gradual, which minimises the risk of kinking, although it is not excluded that there could be a step change in outer diameter of the fibre to produce a more flexible distal portion.

With reference to FIG. 12, this shows another embodiment in which a fibre 12 is provided with a stiffening sleeve or other element 150 along a proximal portion thereof so as to leave a distal portion 152 of the fibre 12 more flexible. The sleeve 150 may have a chamfered end 154 to avoid any hard shoulders within the structure of the apparatus or may have a reducing, tapered, diameter towards the distal end of the fibre 12.

The embodiments of FIGS. 11 and 12 may have any of the light directing elements taught herein.

In practice, a concave axicon lens may be preferable over a convex lens in order to avoid having a focal zone at the tip of the device in the centre of the blood stream when the device is configured to heat the vessel wall. However, where it is desired to heat blood plasma, in some embodiments a convex lens is used to focus light to a focal point beyond the tip 14 of the fibre 12.

The skilled person will also appreciate the precise properties of the light directing element (for instance lens) of the apparatus will be dependent of the choice of the material for the optical fibre.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

The disclosure in the abstract accompanying this application is incorporated herein by reference.

1. Optical energy delivery embolization and/or ablation apparatus for medical applications including:
an optical fibre having a length, a proximal end and a distal end, the distal end including a light directing element configured to direct optical energy for embolization and/or ablation beyond the distal end of the optical fibre;
the proximal end of the optical fibre being configured to be coupled to a source of optical energy;
the optical fibre including a plurality of sensing lattice structures disposed at spaced intervals along the length thereof;
the optical fibre being a combined device for delivery of optical energy and for sensing changes in the optical fibre;
the apparatus including a sensing unit coupled to detect changes in light reflected from the lattice structures and to determine therefrom changes in temperature.

2. Apparatus according to claim 1, wherein the lattice structures are spaced from one another by around 1 mm to around 5 mm.

3. Apparatus according to claim 1, including two or more lattice structures physically positioned so that they either experience same change in temperature with varying strain or same strain with varying change in temperature.

4. Apparatus according to claim 1, wherein the at least one lattice structure is an inscribed micro-lattice structure.

5. Apparatus according to claim 1, wherein the at least one lattice structure is a Bragg grating.

6. Apparatus according to claim 1, wherein the light directing element at the distal end of the optical fibre is or includes a lens.

7. Apparatus according to claim 6, wherein the lens is:
a concave lens;
a concave axicon lens; or
a combination lens in the form of one of: a double convex axicon lens
and a double concave axicon lens.

8. Apparatus according to claim 1, wherein the light directing element at the distal end of the optical fibre is or includes at least one angled surface configured to direct light at an angle out of the distal end of the optical fibre.

9. Apparatus according to claim 8, wherein the light directing element at the distal end of the optical fibre includes at least two angled surfaces configured to direct light in at least two directions out of the distal end of the optical fibre.

10. Apparatus according to claim 8, wherein each angled surface directs light forwardly and radially out of the distal end of the optical fibre.

11. Apparatus according to claim 1, wherein the optical fibre has a varying diameter along its length.

12. Apparatus according to claim 1, wherein the optical fibre has a varying stiffness along its length.

13. Apparatus according to claim 12, wherein the optical fibre has a reducing diameter along its length in the direction of its distal end.

14. Apparatus according to claim 12, wherein the optical fibre includes a stiffening structure in a proximal portion thereof.

15. Apparatus according to claim 1, including a control unit coupled to the source of optical energy and operable to drive the source of optical energy to generate light energy at a plurality of wavelengths.

16. Apparatus according to claim 15, wherein the control unit is operable to utilize different wavelengths for different functions.

17. Apparatus according to claim 16, wherein the control unit is operable to use a first generated wavelength for power transfer.

18. Apparatus according to claim 17, wherein the first wavelength is between 600 nm and 1400 nm.

19. Apparatus according to claim 17, wherein the first wavelength is around 970 nm.

20. Apparatus according to claim 15, wherein the control unit is operable to use a second generated wavelength for sensing.

21. Apparatus according to claim 20, wherein the second wavelength is around 1550 nm or around 800 nm.
